(19) United States
(12) Patent Application Publication
(10) Pub. No.: US 2007/0168221 A1
(43) Pub. Date: Jul. 19, 2007

(54) Communicating medical information

(76) Inventors: James Blotter, Hyde Park, UT (US); Phillip Ray Cowley, Hyde Park, UT (US)

Correspondence Address: R. BURNS ISRAELSEN;Workman Nydegger, 1000 Eagle Gale Tower, 60 E. South Temple, Salt Lake City, UT, 84111, US

(21) Appl. No.: 11/334,595

(22) Filed: Jan. 18, 2006

(51) Int. Cl. G06Q 10/00; A61B 5/00

(52) U.S. Cl. 705/2; 600/300

(57) ABSTRACT

Systems and methods for communicating medical information. Several embodiments also include generating alerts related to medical information, which can be transmitted to different medical entities. Prescription information from a provider describing a medication prescribed for a patient can be received by a management server via the Internet. This prescription information can be communicated from the management server to a pharmacy. Shipment and delivery information is received by the management server. Administration information describing administration of the prescribed medication to a patient is received from a care facility where the medication is administered. This information is stored and accessed by the management server for many purposes, such as inventory, theft prevention, generating alerts, responding to inquiries from governmental, regulatory, and medical entities. Thus, several embodiments provide a new centralized communication point and information storage point for medical entities.

### CROSS-REFERENCE TO RELATED APPLICATIONS

Not applicable

### BACKGROUND OF THE INVENTION

1. The Field of the Invention

The present invention relates to communicating medical information. More particularly, embodiments of the invention relate to tracking prescriptions and associated medications.

2. The Relevant Technology

As the number of medications that are prescribed and administered to patients increases, the management of prescriptions and medications becomes increasingly important. In many instances, people are able to fill their prescription directly with a pharmacist. However, a growing number of patients are cared for by care facilities that may not be located on the same premises as a pharmacy. As a result, delivery of medications and inventory of medications can be substantially error prone and unreliable. This creates many problems for patients, care facilities, pharmacies, and medical providers.

Typically, a prescription for a particular patient at a care facility, such as an assisted living home, is communicated from a provider, such as a doctor, to a pharmacy using a written prescription. The prescription may also be communicated via the telephone or via a network such as the internet. A pharmacist prepares the prescribed medication and the prescribed medication is delivered from the pharmacy to the care facility. At the care facility, the medication is then administered to the patient by the care facility staff.

These conventional methods for both handling prescriptions and delivering the prescribed medications have been based on isolated entities at different destinations. There is often a lack of communication between these medical entities (e.g. medical provider, pharmacist, care facility, and care facility staff) and the delivery and administration processes tend to break down. Moreover, causes of the breakdown cannot be identified to prevent future problems.

The problems that currently exist in handling and delivering prescribed medications may have many adverse effects. For example, a patient may not receive prescribed medication as a result of a missed delivery or theft of their prescribed medication. In addition, a provider may not be made aware that prescribed medication is not being administered to his or her patient. Likewise, a pharmacist may not know that medication is not reaching the care facility or the patient.

There may be no readily accessible way to determine who is responsible for lost or stolen medication. The pharmacist, for example, would not be able to ascertain whether the missed delivery was caused by a mistake or theft at the pharmacy, delivery service, care facility, or care facility staff. Moreover, there may not be sufficient procedures in place at the care facility and delivery service to ensure that the medication is consistently and correctly administered to the patient.

Another factor that impacts the delivery and administration of prescription medication is drug abuse. Some prescribed medications are considered controlled substances, and distribution of such medications at care facilities may be a prime target for those looking to steal these medications. As a result, identification of parties responsible for stolen prescription medication is of interest to many governmental and regulatory organizations and departments. Because tracking medications from the time they leave a pharmacy to the time of administration of the medication to the patient is paper intensive and prone to error and/or fraud, many believe that there is wide spread abuse of prescribed medication. Thus, what would be advantageous is improved communication of medical information such as centralized storage of such information at a single location.

### BRIEF SUMMARY OF SEVERAL EMBODIMENTS

These and other limitations can be overcome by embodiments of the invention, which relate to systems and methods for communicating medical information. In addition to communicating medical information, embodiments of the invention can also generate alerts related to the medical information, which can be transmitted to different medical entities. A method for communicating medical information is disclosed. The method includes receiving prescription information at a management server from a provider describing a medication prescribed for a patient. The method further includes receiving shipment information describing shipment of the prescribed medication at the management server. The method further includes receiving delivery information describing delivery of the prescribed medication at the management server. The method further includes receiving administration information at the management server describing administration of the prescribed medication to the patient. The method further includes associating the prescription information, the shipment information, the delivery information, and the administration information to manage the distribution of the prescribed medication to the patient.

A method for communicating administration information that describes administration of prescription medication to a patient is disclosed. The method includes transmitting delivery information indicating that a prescribed medication for a patient at a care facility has been delivered to the care facility. The method further includes receiving a signal from a handheld device, the signal providing a real-time indication whether the patient has been administered the prescribed medication. The method further includes transmitting administration information describing whether the patient was administered the prescribed medication.

A method for tracking medication from prescription of the medication by a provider to administration of the medication to a patient is disclosed. The method includes receiving prescription information from a provider that generates a prescription identifying medication for a patient. The method further includes receiving shipment information from a pharmacy that fills the prescription for the patient, the shipment information describing shipment of the medication to a care facility. The method further includes receiving delivery information from a carrier that delivers the medication from the pharmacy to a care facility, the delivery information describing delivery of the medication to the care facility. The method further includes receiving administration information from the care facility, the administration information describing administration of the medication to the patient. The method further includes analyzing the prescription information, the shipment information, the delivery information, and the administration information to generate alerts for one or more of the provider, the pharmacy, the care facility or the patient, wherein the alerts are used for at least one of: controlling inventory of the medication at the pharmacy or the care facility; identifying whether conditions of the prescription are satisfied at the care facility; determining whether the medication was delivered to the care facility; and advising of a recall for the medication. These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates a block diagram of a system for managing the distribution of medications and prescriptions;

FIG. 2 is a block diagram illustrating a method for communicating medical information;

FIG. 3 is a block diagram illustrating a method for communicating medical information;

FIG. 4 is a block diagram illustrating a method for generating an alert;

FIG. 5 is a block diagram illustrating a method for generating an alert;

FIG. 6 is a block diagram illustrating a method for generating an alert based on third party medical information; and

FIG. 7 illustrates an example of a data processing system, such as a computer terminal located at a provider, pharmacy, care facility, and/or management server.

### DETAILED DESCRIPTION OF THE PREFERRED EMBODIMENTS

Embodiments of the invention relate to communicating medical information. The communication of medical information is achieved using a management server with centralized storage capability. Each entity involved in the prescription and delivery of medications is able to communicate with the management server. As each entity performs certain tasks, the results are communicated to the management server and stored. This management server can therefore generate a timeline that identifies what happened to specific medications. Further, the management server can generate alerts that are used to identify and correct errors that may occur in the delivery and/or administration of prescription medications.

It should be understood that the drawings are diagrammatic and schematic representations of such example embodiments and, accordingly, are not limiting of the scope of the present invention, nor are the drawings necessarily drawn to scale. Well known devices and processes have been excluded so as not to obscure the discussion in details that would be known to one of ordinary skill in the art.

1. Examples of Systems for Communicating Medical Information

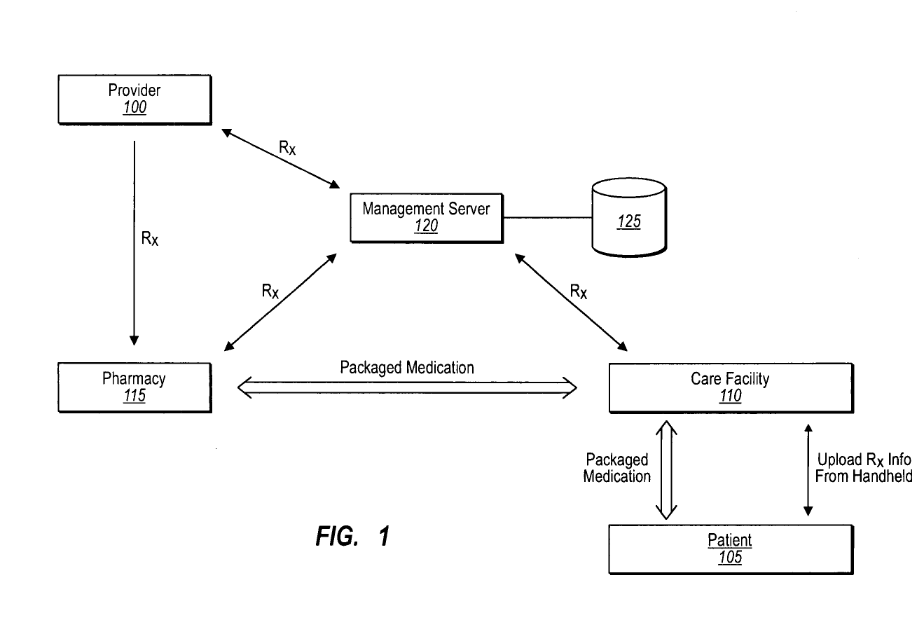

FIG. 1 is a block diagram illustrating exemplary systems and methods for communicating medical information including managing the distribution and/or administration of medications and prescriptions. In addition to managing the distribution of medications and prescriptions, the system illustrated in FIG. 1 also illustrates a management server that provides a centralized storage of medical information. This medical information communicated to, and stored by, the management server can describe the tracking, distribution, recall, inventory information, diagnosis information, counseling information, formulary information, and/or administration of the medications and prescriptions, as well as other medical information.

A provider 100, such as a doctor, provides a prescription for a patient 105 cared for at a care facility 110. Examples of care facility 110 include assisted living homes and other facilities which provide medical care for patients, but do not have on-site pharmacies. The provider 100 provides the prescription to a pharmacy 115. The provider 100 can also, or alternatively, provide the prescription information to a management server 120.

The management server 120 receives the prescription information from the provider 100 and stores the prescription information in a database 125. The management server 120 can receive the prescription information from the provider 100 via a network, and the network can include the Internet. The management server 120 can be part of a third party service that is not affiliated with any of the provider 100, pharmacy 115, or the care facility 110. According to alternative embodiments, the management server 120 can be associated with the provider 100, pharmacy 115, the care facility 110, and/or a regulatory or governmental agency.

A distinct account for the patient 105 can be created at the management server 120 by an authorized party. For example, the provider 100 can provide the management server 120 with unique patient 105 and care facility 110 information prior to providing the prescription to the management server. In addition, the provider 100, or the care facility 110 can associate a particular pharmacy 115 with the particular care facility 110 or patient 105. Thus, the care facility 110, patient 105, and pharmacy 115 can be associated within the database 125 within a unique account, and the database 125 can receive updates to these associations from the care facility 110, pharmacy 115, and/or provider 100.

The management server 120 receives the prescription from the provider 100 and transmits the prescription to the pharmacy 115. The management server 120 can manage the distribution of prescriptions to several pharmacies at different locations. The management server 120 can also store information describing medical records, diagnosis, and impressions received from the provider 100. Thus, in addition to managing the distribution of prescribed medication, the management server can provide the pharmacy 115, care facility 110, provider 100, patient 105, or other entity (such as a regulatory or law enforcement agency) access to any of this information where the requesting party has proper authorization to receive the information.

Electronic counseling (e.g. from a pharmacist) and patient education information may be continuously available for each medication supplied for the patient 105. The patient education information can be made available through the management server 120 via a network such as the internet. Images of medications and descriptions of their packaging may be available for the patient 105.

Upon receiving the prescription for the patient 105 from the provider 100, the pharmacy 115 packages the prescribed medication for delivery to the care facility 110. The pharmacy 115 can package the medication in customized blister packs, for example, for the particular patient 105. The customized blister packs can be configured with individual compartments for particular administration periods or for different types of medication. Other types of packaging can also be used as known to one of skill in the art.

The medication packs can have readable indicia for tracking the medication packs and/or association of the medication packs. For example, the medication packs can include optical machine readable indicia, such as a barcode, for electronic tracking of the medication packs. The indicia may be printed on the medication packs and can be associated with the medication contained within the packs, the care facility 110, the pharmacy 115, the prescription, a care facility staff member, and/or the patient 105 to which the medication is to be administered. The indicia on the medication packs can also encode this information such that the information can be decoded directly from the indicia. In some instances, the medication packs are customized.

The management server 120 can be integrated with equipment and systems that automatically fill prescriptions. Various automated prescription filling systems currently exist, and the management server 120 may integrate with these systems to provide data for automated prescription filling. Automated prescription packaging systems may exchange electronic data from the pharmacy 115, the provider 100, or the care facility 110 through the management server via a network such as the internet.

Next, the prescribed medication is delivered to the care facility 110. The prescribed medication is tracked at the time it ships from the pharmacy 115 and at the time that it is delivered to the care facility 110. Information describing the shipment and delivery of the medication can be transmitted to the management server 120 via the Internet or other suitable network. The indicia on the medication packs can be read by an optical reader at the pharmacy 115 and the care facility 110 to electronically track the medication. The medication packs can also be grouped into larger sets of packs and further packaged within a container, such as a box or sack. Each of the medication packs can be associated with the larger set and container. The larger set and container can also include additional indicia associating the set and container with a particular care facility 110, for example. Tamper identification means, such as tape or a closure device, can be used to indicate whether the container of medication packages has been tampered with between departure from the pharmacy 115 and arrival at the care facility 110.

Upon receiving the prescribed medication, the care facility 110, or a carrier, sends an indication to the management server 120 that the prescribed medication was delivered from the pharmacy 115. Upon receiving the delivery indication from the care facility 110, the management server 120 stores information in the database 125 describing the delivery of the medication to the care facility 110. This information can be associated with information received from the pharmacy 115 regarding shipment of the medication from the pharmacy 115. The shipment and delivery information transmitted and stored at the management server can include a description of the time of shipment and time of delivery and can also be associated with information describing the patient 105, care facility 110, and/or pharmacy 115. This information can be accessed and used for analysis and/or generating subsequent reports, for example.

After receiving the prescribed medication, the care facility 110 distributes the medication to the patient 105. Information describing the manner in which the medication is to be distributed can be encoded in the indicia on the medication pack. Information describing the manner in which the medication is to be distributed can also be downloaded from the management server 120, for example by sending a request to the management server 120 referencing the particular medication pack.

The patient 105 can also be associated with a computer-readable indicia at the care facility 110. For example, the patient 105 can be associated with a patient associated barcode and the care facility staff can scan both the indicia of the medication and the patient's 105 barcode at the time that the medication is administered to the patient.

It should be appreciated, however, that patient-prescription information may be obtained automatically by a bar code scanner and hand held device, input manually from a terminal at the care facility, or by another means for tracking administration of medication.

Once the medication is distributed to the patient 105 (or refused by the patient), information describing the administration of the medication (or information describing a refusal of administration of the medication) is transmitted to the management server 120. This information can be collected by handheld units or entries into computer terminals by care facility staff. This information can include the type of medication administered, the amount of medication administered, the time of administration of the medication, and/or whether the patient took the medication. The administration information can be transmitted to the management server 120 via the Internet. For example, once the medication is administered, the care facility staff can scan a bar code, or other indicia, on the medication pack and information indicating administration of the medication. The administration information can be automatically and electronically sent to the management server 120 via the Internet. Upon receiving the administration information, the management server 120 stores the administration information in the database 125 along with, and associated with, the other information related to the patient, prescription, and medication.

According to this embodiment, at this point the management server 120 has stored a real-time, or near real-time, historical timeline tracking the medication from the time that it was prescribed by the care provider 100, left the pharmacy 115, was delivered to the care facility 110, and was administered to the patient 105. In addition, the database 125 includes a centralized location for accessing the information describing all of these events.

Tracking medication provides many benefits not available under conventional methods. For example, real time tracking of medication can provide evidence of theft of the prescribed medication for law enforcement agencies. The tracking will also likely deter would-be thieves from stealing the medication if they know that the medication is being tracked. The information can also be readily available for access at the single location of the management server 120 making comparison of the information much easier for many purposes.

The information stored at the management server 120 can be used for inventory purposes. For example, the pharmacy 115 can monitor the amount of medication stored at the care facility 110 along with the needs of the patient 105 at the care facility. This information can be used to anticipate the needs of the care facility 110 for new medications. Therefore the pharmacy 115 may not need to keep as much medication on-hand and can order the medication in anticipation of the needs of the care facility 110 (along with other care facilities) thereby insuring that the pharmacy 115 has the needed medications without unduly burdening the pharmacy with stocks of medications until they are needed. Thus, the medications can be ordered “just-in-time.”

Inventory control information and medication orders may be submitted from the pharmacy directly to wholesale medication providers or other parties via the management server 120. Medications, packaging, or any other relevant item may be requested directly through the management server 120 via a network such as the internet.

Many inventory aspects can also be accomplished using alerts and reports generated by the management server 120 for the pharmacy 115 indicating when the care facility 110 is likely to need medication. For example, when inventory of a medication at the care facility 110 reaches a threshold level (e.g. 10-40% of stock) an alert can be sent to the pharmacy 115 indicating that an order for the medication will likely need to be filled in the near future such that the pharmacy 115 has enough medication in time for the potential order.

Real-time tracking of the medication can also provide a service to the provider 100 to monitor the status of the patient 105 (as well as other patients). For example, the provider 100 can receive status reports and alerts regarding the patient 105. These alerts and status reports can indicate whether the patient 105 is receiving medications appropriately and according to the provider's 100 prescription. Additional information can be transmitted from the care facility 100 to the management server 120 regarding the patient 105, such as impressions of the care facility staff, adverse reactions, and other developments, which can be stored at the management server 120 and accessed by the provider 100 or any authorized party. Thus, the management server 120 enables the provider 100 to receive information regarding their patients and real time data describing the medications which the provider 100 prescribed.

2. Examples of Methods for Communicating Medical Information

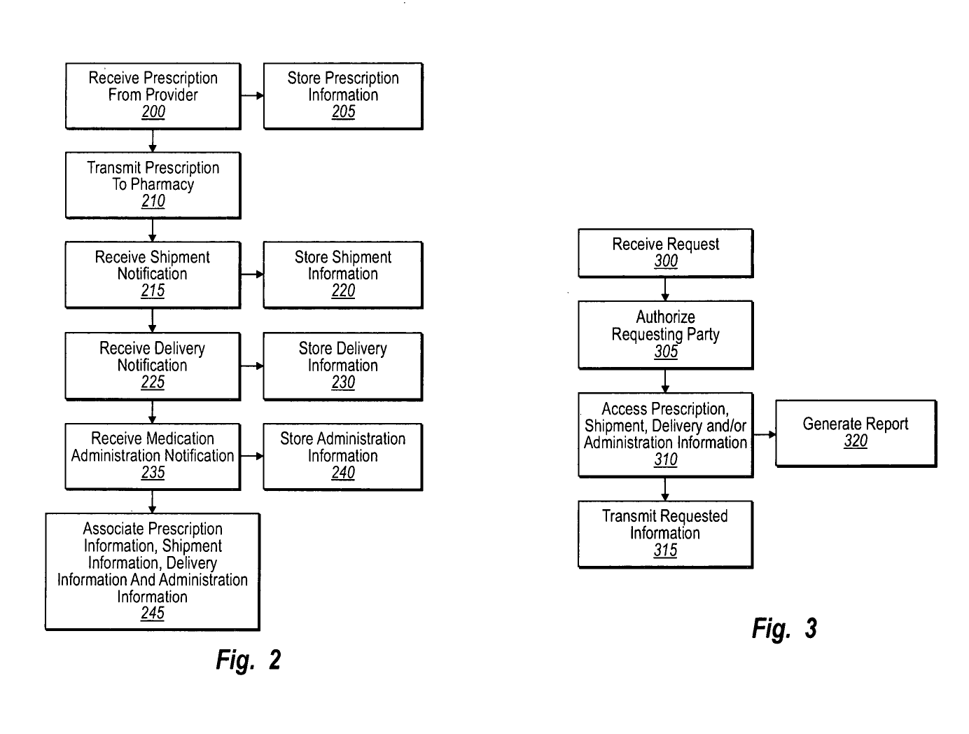

FIG. 2 illustrates one embodiment of a method for communicating medical information. A prescription is received 200 from a provider, for example at a management server. The provider can be a doctor, physician, or other medical personnel and the prescription can be received electronically via a network including the Internet. The prescription can be received by the management server via an online graphical user interface located on a web page hosted by the management server. Once the prescription is received by the management server, the management server stores 205 the prescription in a computer-readable medium. The computer-readable medium can be any type of computer-readable medium and can be local to the management server or can be accessible to the management server via a network, which can include the Internet.

The management server transmits 210 the prescription to a pharmacy. In the instance that the provider communicates the prescription to the pharmacy this step may not be necessary. The prescription is received by the pharmacy and the pharmacy prepares the prescription for delivery to a care facility. The prescription can include information such as the location of the care facility and other information needed for the pharmacy to identify the patient and deliver the medication to the care facility.

The prescribed medications can be uniquely packaged by the pharmacy. For example, the medications can be packaged in a blister package or other suitable packaging that is configured to store or sort the medication. The medications in the blister pack can be uniquely packaged for a particular patient. The pack can also be uniquely configured according to a predefined medication administration scheme. For example, the package can include different compartments for different medications depending on the time for administration of the medication to the patient according to the prescription or type of medication. The package of medication can include indicia that is used in tracking, delivering, and/or administering the prescribed medications. For example, the indicia can be a barcode or other indicia that may be readable by an optical scanner. The indicia can be used to track the medication pack from the time that it leaves the pharmacy to the time that it is administered to the patient.

A shipment notification is received 215 when the pharmacy ships the medication. The shipment notification indicates that the prescribed medication has been shipped by the pharmacist. The shipment may be carried out by an employee of the pharmacy, can be a carried out by common carrier, or can be carried out by other means for transporting the medication to the care facility. The shipment notification can be an electronic notification of the shipment and can be received as the medication leaves the pharmacy or as the medication is taken by the carrier. The shipment notification can be transmitted to the management server by the pharmacy, carrier, or by another party indicating that the medication has left the pharmacy and/or has been received by the carrier.

Information describing the shipment is stored 220 in a computer-readable medium. The information stored by the management server can describe the time and date of the shipment. The information can also include a description of the carrier, such as the person receiving and responsible for the medication delivery. The information can include a description of each person who will handle the pack of medication before it is delivered to the care facility.

A delivery notification, transmitted by the carrier or the care facility, is received 225 when the medication is delivered to the care facility. Information describing the delivery is stored 230 in the computer-readable medium at the management server. The information stored can indicate the time and day on which the medication was delivered to the care facility. The information can also include a description of any abnormality or tampering with the medication. For example, the package can include an anti-tamper device, such as a tape or mechanism, which indicates whether the medication pack, or a container containing the medication pack, has been tampered with. The delivery information can also include a description of the medication and a description of the condition of the medication when it was delivered. The delivery information can describe the facility to which the medication was delivered and the intended patient to which the medication is to be administered.

After the prescribed medication is delivered to the care facility it is distributed and administered to the patient according to the prescription. The medication can be administered by care facility staff who can record when the medication is administered. For example, the care facility staff can use handheld units to indicate when the medication is administered to the patient. The handheld units can include a scanner for reading indicia on the packs. The scanner can be a barcode reader and the care facility staff can scan a barcode on the pack indicating that the medication is being administered. Each patient can also have patient indicia associated with the patient. The patient indicia can be a barcode or other scannable indicia, which can be scanned along with the medication when the medication is administered. Administration information can also be collected by other means.

Notification that the medication has been administered is transmitted by the care facility and received 235 at the management server. The notification can describe the medication administered and the patient to which the medication was administered. Administration information describing the administration of the medication is stored 240 in a computer-readable medium by the management server. The administration information can indicate a time and date on which the medication was administered and can include a description of the patient to which the medication was administered to and any other information describing the administration of the medication to the patient. The medication administration information can be acquired using a handheld unit as the medication is administered to the patient. The administration information can be transmitted by the handheld unit to a care facility terminal and subsequently from the care facility terminal to the management server via the Internet. The administration information can also be entered directly into a terminal and transmitted to the management server via the Internet by the terminal. The administration information can also include comments or impressions of the care facility staff administering the medication.

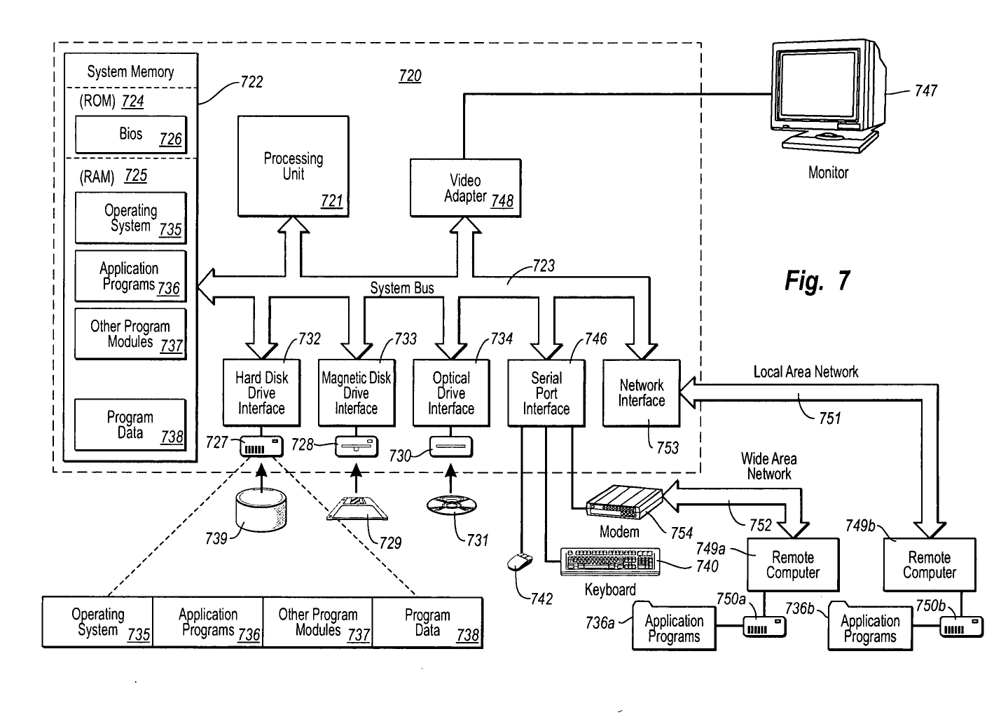

This administration information can be compared with other information, such as the prescription, to generate alerts, which can be transmitted to the care facility provider, pharmacy, and/or a governmental or regulatory agency. The administration notification can be transmitted from the handheld unit to a care facility server (e.g. including a terminal illustrated in FIG. 7) and the notification can be transmitted from the care facility server or terminal to the management server via the Internet. A terminal can also be used to manually enter administration information and transmit the administration notification to the management server rather than using the handheld units.

Referring to FIG. 3, a block diagram illustrating a method for communicating medical information to an authorized party is shown. Advantageously, the medical information has been collected at the management server and is therefore available and accessible to authorized parties. This enables an authorized party to view the history of a patient and/or prescription, for example, from the time the prescription is issued by a provider until the prescribed medication is administered to the patient. This historical view of the medical information can identify weaknesses in the delivery and/or administration of medication and can also be used to alert the various entities involved in the delivery and administration of medication to various issues or problems in a timely manner.

In this example, a request for medical information is received 300 at a management server. Typically, communications with the management server occur over the Internet or other appropriate network. The request can be received from a medical provider, a pharmacy, a care facility, a regulatory agency, a governmental agency, or other interested party. Authorization for the requesting party to receive the information can be verified 305. Verification of the requesting party can be accomplished using a logon and a password or using other means, which can be also received along with, or separate from, the request.

The requested information is accessed 310 from a database. The database can include patient information, prescription information, shipment information, delivery information, administration information, provider information, pharmacist information, carrier information, care facility information, care facility staff information, recall information, diagnosis information and/or other medical information related to the prescribed medication and/or the patient. This information, on a per patient or other basis, can be accumulated over time.

The requested information is transmitted 315 to the requesting party, if authorized. A report can also be generated 320 regarding the requesting party, the patient, medications, and/or other requested information. The report can include the requested information and can be exported to a printer. The information requested can be processed and organized according to several criteria. The report can be generated in response to a request by the Joint Commission of Accreditation of Health Care Facilities (JCAHO) or to satisfy a requirement of the JCAHO or other commission, organization, or agency.

In response to a request for information or for any other reasons, the information stored by the management server can be processed to determine if there are any inconsistencies or violations of conditions. Conditions may include, by way of example, incorrect delivery of medication, misadministration of medication, violation of a condition of a prescription, or other violation of a condition. The conditions can be based on regulations or laws, such as regulations and laws governing controlled substances. A condition can be a condition set forth by the JCAHO.

3. Examples of Methods for Generating Alerts

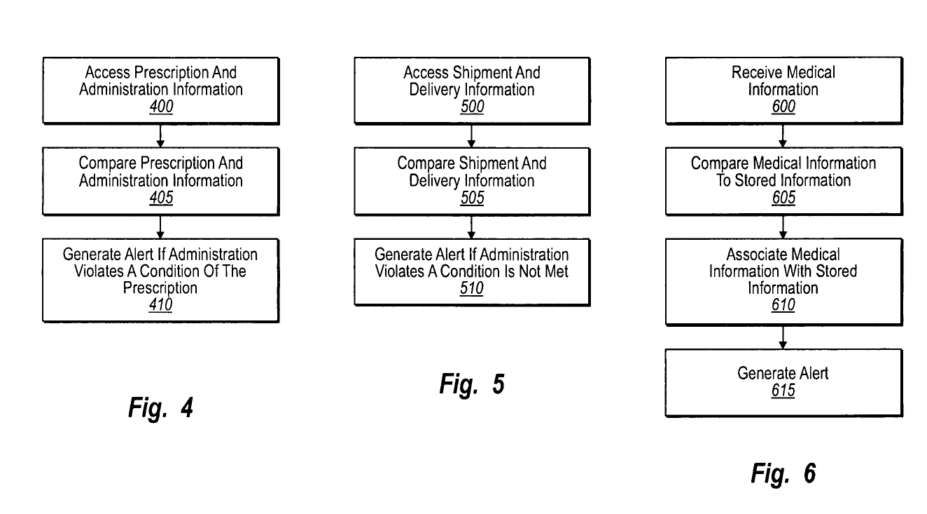

Referring to FIG. 4, a block diagram illustrating a method for generating an alert is shown. FIG. 4 illustrates an exemplary process for generating an alert when the conditions of a prescription are not followed. The generation of an alert typically begins by accessing 400 prescription and administration information from a database of the management server. As previously described, the database of the management server stores medical information, which includes prescription and administration information.

In this case, the prescription information (received from a provider) is compared 405 to the administration information (received, for example, from a care facility). An alert is generated 410 in the instance that the administration of the medication to a patient violates a condition of the prescription. For example, an alert can be generated in the instance that the medication was administered to the wrong patient, at the wrong time, on the wrong day, in a wrong amount, by a wrong or unqualified person, was not administered (e.g. refused by the patient), or according to any other criteria. The alert can be transmitted to the provider, pharmacy, care facility, care facility staff, law enforcement or regulatory agency, or any other party.

Referring to FIG. 5, a block diagram illustrating a method for generating an alert is shown. In this case, the generated alert relates to the shipment of medication from a pharmacy and the delivery of the prescribed medication to a care facility. This example begins by accessing 500 shipment and delivery information from the management server's database. The shipment information (generated by the pharmacy, for example) is compared 505 to the delivery information (generated at the care facility, for example).

In the instance that the comparison of the shipment information to the delivery information indicates that there is a violation of a condition then an alert is generated 510. One condition that generates the alert is when medication that was shipped was not delivered. The condition can also indicate whether the medication was shipped or delivered on time. The condition can also indicate whether the packaging of the medication has been tampered with. In other words, the shipping information can be compared with the delivery information to determine whether the medications were successfully shipped and delivered. The conditions are designed to identify situations where a problem occurred either in the shipping of the medication or the delivery of the medications. The alert can describe a result of the comparison between the information received from the various entities at the management server.

In addition, the alert can also describe a party responsible for the violation. For example, the alert can indicate who was responsible for the medication when the violation was performed. It should be appreciated that one or more of the shipping, delivery, prescription, provider, and administration information stored by the management server may need to be accessed to generate such an alert. Information describing alerts can also be associated with any of the entities described herein such that they can be accessed from the computer-readable medium based on an inquiry.

Additional information received from third parties can also be communicated to the management server, provider, pharmacy, care facility, care facility staff, and/or the patient. For example, a recall from a manufacturer or agency, such as the Federal Drug Administration (FDA), can be communicated to the management server, and subsequently from the management server to the provider, pharmacy, care facility, care facility staff, and/or patient. This communication of a recall can be communicated via the Internet. Thus, the embodiments disclosed herein can enable communication between third parties and the medical entities, which can provide information to the management server via an online website. As a result, each entity can remove recalled medications from storage and/or administration rotations thereby expediting communication of such medical information from third parties to providers, pharmacists, care facilities, staff, and patients.

Third party information can also be associated with particular medications, prescriptions, treatments, care facilities, and patients at the management server and can be used to generate alerts. For example, referring to FIG. 6, a block diagram illustrating a method for generating an alert based on third party medical information is shown. This example begins by receiving 600 medical information. The medical information can relate to a prescription medication. The medical information can be received from a manufacturer of the medication or an agency for regulating medication, such as the FDA. For example, the medical information can be a recall of a medication.

The medical information is compared 605 to information stored in a database of the management server. As previously stated, the information stored in the database can include prescription information for a patient, shipment of medication information, delivery of the medication information, and/or administration information. The database can also include stored information describing diagnosis, observations, and/or additional information describing the patient or the medication prescribed to the patient.

The medical information received from the third party is associated 610 with information stored in the database. For example, where the medical information is a recall of the medication, the recall of the medication can be associated with providers prescribing the medication, pharmacies having the medication in stock, care facilities distributing the recalled medication, care facility staff administering the prescribed medication, and/or patients who are being administered the recalled medication.

An alert is generated 615 based on the received information and its comparison to existing information in the database. The alert can be transmitted to a provider, pharmacy, care facility, care facility staff and/or patient. The alert can include information describing a recall of medication, for example. As a result of receiving the alert, a provider can discontinue prescribing the recalled medication, a pharmacy can discontinue shipping the recalled medication, a care facility and staff can discontinue distributing and administering the recalled medication and a patient can discontinue taking the recalled medication.

A recall, or other medical information, can also be stored in the database and alerts can be generated if the recalled medication is prescribed, shipped, delivered, distributed, or administered. Thus, additional medical information can be stored in the database, accessed by the management server, and compared to other information in the database, or information received by the management server to generate additional alerts and communicate these alerts to providers, pharmacies, care facilities, and/or patients. Any of these alerts, and information describing communication of these alerts to providers, pharmacies, care facilities, and/or patients can be stored in memory, such as the database, and accessed by the management server, for example to provide ongoing and archived information on medication errors and/or transmission of information describing medication errors.

Formulary control may be available through the management server. Current formulary status for any purpose will be available to the pharmacist, the provider, and the care facility. The management server may provide an alert to the pharmacist, the provider, or the care facility, when a formulary exchange is possible.

With reference again to FIGS. 1 and 7, data processing systems at the management server 120, provider 100, pharmacy 115, and care facility 110 can include computer-readable medium for carrying or having computer-executable instructions or data structures stored thereon. Such computer-readable medium can be any available media that can be accessed by a general purpose or special purpose computer. By way of example, and not limitation, such computer-readable medium can include RAM, ROM, EEPROM, CD-ROM or other optical disk storage, magnetic disk storage or other magnetic storage devices, millipede and other Micro Electro-Mechanical Systems (MEMS), or any other device or medium which can be used to carry or store desired program code means in the form of computer-executable instructions or data structures. Such computer readable medium can be accessed by a general purpose or special purpose computer. When information is transferred or provided over a network or another communications connection (either hardwired, wireless, or a combination of hardwired or wireless) to the provider 100, pharmacy 115, management server 120, and care facility 110, the connection is properly viewed as a computer-readable medium. Thus, any such connection is properly termed a computer-readable medium. Combinations of the above should also be included within the scope of computer-readable medium.

Computer-executable instructions comprise, for example, instructions and data which cause a processing device to perform a certain function or group of functions and data. Data stored on a computer-readable medium can properly be referred to by the information which the data represents when presented to a user or processed by a computer.

Although not required, several embodiments discussed herein are carried out by computer-executable instructions, such as program modules, being executed by computers in the network environments. Generally, program modules include routines, programs, objects, components, data structures, etc. that perform particular tasks or implement particular abstract data types. Computer-executable instructions, associated data structures, and program modules represent examples of the program code means for executing steps of the methods disclosed hereinafter. The particular sequence of such executable instructions or associated data structures represents examples of corresponding acts for implementing the functions described in such steps.

Those skilled in the art will appreciate that the embodiments illustrated herein may be practiced in network computing environments with many configurations, including personal computers, handheld devices, multi-processor systems, portable devices, microprocessor-based devices, programmable consumer electronics, network PCs, minicomputers, mainframe computers, and the like. Several embodiments may also be practiced in distributed environments where tasks are performed by local and remote processing devices that are linked (either by hardwired links, wireless links, or by a combination of hardwired and wireless links) through a communications network. In a distributed environment, program modules may be located in both local and remote memory storage devices. Multiple management servers 120 can also be implemented and can communicate directly or across a network with multiple providers 100, multiple pharmacies 115, and/or multiple care facilities 110 for administration of medication to multiple patients 105.

With reference to FIG. 7, an example of a data processing system, such as a computer terminal located at the provider 100, pharmacy 115, care facility 110, or management server 120 of FIG. 1 is illustrated for implementing several embodiments discussed herein. The computer terminal is illustrated as a general purpose computing device in the form of a conventional computer 720. The computer 720 includes a processing unit 721, a system memory 722, and a system bus 723 that couples various system components including the system memory 722 to the processing unit 721. The system bus 723 may be any of several types of bus structures including a memory bus or memory controller, a peripheral bus, and a local bus using any of a variety of bus architectures. The system memory includes read only memory (ROM) 724 and random access memory (RAM) 725. A basic input/output system (BIOS) 726, containing the basic routines that help transfer information between elements within the computer 720, such as during start-up, may be stored in ROM 724.

The computer 720 may also include a magnetic hard disk drive 727 for reading from and writing to a magnetic hard disk 739, a magnetic disk drive 728 for reading from or writing to a removable magnetic disk 729, and an optical disk drive 730 for reading from or writing to removable optical disk 731 such as a CD-ROM or other optical media. The magnetic hard disk drive 727, magnetic disk drive 728, and optical disk drive 730 are connected to the system bus 723 by a hard disk drive interface 732, a magnetic disk drive-interface 733, and an optical drive interface 734, respectively. The drives and their associated computer-readable medium provide nonvolatile storage of computer-executable instructions, data structures, program modules and other data for the computer 720. Although the exemplary environment described herein employs a magnetic hard disk 739, a removable magnetic disk 729 and a removable optical disk 731, other types of computer-readable medium for storing data can be used, including magnetic cassettes, flash memory cards, digital versatile disks, Bernoulli cartridges, RAMs, ROMs, and the like. The database 125 of FIG. 1, for example, can be any computer-readable medium in the computer 720, or coupled to the computer 720 as computer-readable medium local to, or remotely coupled to, the computer 720.

Program code means comprising one or more program modules may be stored on the hard disk 739, magnetic disk 729, optical disk 731, ROM 724 or RAM 725, including an operating system 735, one or more application programs 736, other program modules 737, and program data 738. A user may enter commands and information into the computer 720 through keyboard 740, pointing device 742, or other input devices (not shown), such as a microphone, joy stick, game pad, satellite dish, scanner, wireless device, or the like. These and other input devices are often connected to the processing unit 721 through a serial port interface 746 coupled to system bus 723. Alternatively, the input devices may be connected by other interfaces, such as a parallel port, a game port or a universal serial bus (USB). A monitor 747 or another display device is also connected to system bus 723 via an interface, such as video adapter 748. In addition to the monitor, personal computers typically include other peripheral output devices (not shown), such as speakers and printers.

The computer 720 may operate in a networked environment using logical connections to one or more remote computers, such as remote computers 749a and 749b. Remote computers 749a and 749b may each be another personal computer, a server, a router, a network PC, a peer device or other network node, and can typically include any or all of the elements described above relative to the computer 720, although only memory storage devices 750a and 750b and their associated application programs 736a and 736b have been illustrated in FIG. 7. The logical connections depicted in FIG. 2 include a local area network (LAN) 751 and a wide area network (WAN) 752 that are presented here by way of example and not limitation. Such networking environments are commonplace in office-wide or enterprise-wide computer networks, intranets and the Internet.

When used in a LAN networking environment, the computer 720 is connected to the local network 751 through a network interface or adapter 753. When used in a WAN networking environment, the computer 720 may include a modem 754, a wireless link, or other means for establishing communications over the wide area network 752, such as the Internet. The modem 754, which may be internal or external, is connected to the system bus 723 via the serial port interface 746. In a networked environment, program modules depicted relative to the computer 720, or portions thereof, may be stored in the remote memory storage device. It will be appreciated that the network connections shown are examples and other means of establishing communications over wide area network 752 may be used.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. A method for communicating medical information, the method comprising:
receiving prescription information at a management server from a provider describing a medication prescribed for a patient;
receiving shipment information describing shipment of the prescribed medication at the management server;
receiving delivery information describing delivery of the prescribed medication at the management server;
receiving administration information at the management server describing administration of the prescribed medication to the patient; and
associating the prescription information, the shipment information, the delivery information, and the administration information to manage the distribution of the prescribed medication to the patient.

2. A method according to claim 1, further comprising storing the prescription information, shipment information, delivery information, and administration information in a computer-readable medium accessible by the management server.

3. A method according to claim 1, wherein the administration information includes real-time information describing whether the patient took the prescribed medication or refused the medication.

4. A method according to claim 1, further comprising transmitting the prescription information from the management server to a pharmacy.

5. A method according to claim 1, wherein the shipment of the prescribed medication includes a patient medicine pack storing different medications for the patient according to the prescription of the patient.

6. A method according to claim 5, wherein the patient medicine pack includes indicia for identifying the patient to which the medicine pack is prescribed.

7. A method according to claim 6, wherein the indicia is a barcode and the barcode further indicates the care facility at which the patient is located and a time at which the prescribed medication is to be administered to the patient.

8. A method according to claim 1, wherein the administration information is acquired using a handheld unit at a care facility, wherein the handheld unit records a time at which the patient is administered the prescribed medication.

9. A method according to claim 1, further comprising:
receiving a request from the provider for the administration information for the patient; and
transmitting the administration information for the patient to the provider.

10. A method according to claim 1, further comprising:
receiving a request for at least one of the shipping information, delivery information, prescription information, or administration information from a law enforcement agency, an administrative agency, or a government agency; and
transmitting the requested information to the law enforcement agency, administrative agency, or government agency.

11. A method according to claim 1, wherein associating the prescription information, the shipment information, the delivery information, and the administration information to manage the distribution of the prescribed medication to the patient further comprises:
comparing the prescription information to at least one of the administration information or the shipment information to the tracking information; and
selectively generating an alert based on a result of the comparison.

12. A method according to claim 11, further comprising comparing one or more of the prescription information, the shipment information, the delivery information, or the administration information using conditions to detect violations of the conditions, wherein the conditions include one or more of incorrect delivery of medication, misadministration of medication, violation of a condition of a prescription, loss of the prescribed medication, or failure to ship the prescribed medication.

13. A method according to claim 11, further comprising: if the alert is generated, transmitting the alert to at least one of the provider, the pharmacy, the care facility, the patient, a law enforcement agency, a regulatory agency, or a government agency.

14. A method according to claim 1, further comprising:
receiving recall information at the information server; and
transmitting the recall information to at least one of the provider, a pharmacy, a care facility, care facility staff, or a patient.

15. A method according to claim 1, further comprising:
transmitting formulary status information to at least one of the pharmacist, provider, or care facility.

16. A method for communicating administration information that describes administration of prescription medication to a patient, the method comprising:
transmitting delivery information indicating that a prescribed medication for a patient at a care facility has been delivered to the care facility;
receiving a signal from a handheld device, the signal providing a real-time indication whether the patient has been administered the prescribed medication; and
transmitting administration information describing whether the patient was administered the prescribed medication.

17. A method according to claim 16, wherein receiving a signal from a handheld device further comprises acquiring the signal with a scanner on the handheld device that reads a barcode on an individual pack of medication as the medication is administered to the patient.

18. A method according to claim 17, wherein receiving a signal from a handheld device further comprises scanning a barcode associated with the patient such that the patient is associated with the barcode on the individual pack of medication.

19. A method according to claim 17, further comprising managing delivery of the prescription medication to the patient by performing one or more of:
receiving prescription information at a management server, the prescription information describing a prescription for the patient from a network including the Internet;
transmitting the prescription information from the management server to a pharmacy via the network;
receiving shipment information at the management server from the pharmacy describing shipment of the prescription;
receiving the delivery information at the management server, the delivery information describing receipt of the prescription at the care facility from the network including the Internet;
receiving the administration information at the management server from the network including the Internet; and
storing the prescription information, shipment information, tracking information, and administration information in a computer-readable medium.

20. A method according to claim 19, further comprising:
performing the following at a pharmacy:
receiving the prescription information from the management server;
shipping the prescribed medication to a care facility; and
transmitting shipment information describing shipment of the prescribed medication to the management server.

21. A method according to claim 19, further comprising:
receiving a request at the management server from an authorized entity for a set of stored administration information, the set of stored administration information including administration information for a requested time period; and
transmitting the set of stored administration information to the authorized entity.

22. A method according to claim 21, further comprising:
receiving a request at the management server via the network from a regulatory, governmental, or law enforcement agency for one or more of the prescription information, shipment information, tracking information, or administration information; and
transmitting the requested information via the network to the regulatory, governmental, or law enforcement agency.

23. A method according to claim 22, wherein the requested information is requested by the Joint Commission of Accreditation of Health Care Facilities (JCAHO), the method further comprising generating a report to satisfy a requirement set forth by the JCAHO

24. A method for tracking medication from prescription of the medication by a provider to administration of the medication to a patient, the method comprising:
receiving prescription information from a provider that generates a prescription identifying medication for a patient;
receiving shipment information from a pharmacy that fills the prescription for the patient, the shipment information describing shipment of the medication to a care facility;
receiving delivery information from a carrier that delivers the medication from the pharmacy to the care facility or receiving the delivery information from the care facility that receives the medication from the carrier, the delivery information describing delivery of the medication to the care facility;
receiving administration information from the care facility, the administration information describing administration of the medication to the patient;
analyzing the prescription information, the shipment information, the delivery information, and the administration information to generate alerts for one or more of the provider, the pharmacy, the care facility or the patient, wherein the alerts are used for at least one of:
controlling inventory of the medication at the pharmacy or the care facility;
identifying whether conditions of the prescription are satisfied at the care facility;
determining whether the medication was delivered to the care facility; and
advising of a recall for the medication.

25. A method according to claim 24, wherein analyzing the prescription information, the shipment information, the delivery information, and the administration information to generate alerts for one or more of the provider, the pharmacy, the care facility or the patient further comprises comparing the prescription information with the administration information or comparing the shipment information with the delivery information.

26. A method according to claim 24, further comprising generating one or more of the shipment information, the delivery information, or the administration information by scanning indicia associated with the medication, wherein the indicia represents information associated with the medication.

27. A method according to claim 24, wherein identifying whether conditions of the prescription are satisfied at the care facility further comprises one or more of:
generating an alert if the medication was administered to a wrong patient;
generating an alert if the medication was not administered to the patient;
generating an alert if the medication was not delivered to the care facility;
generating an alert if the medication was delivered in a wrong amount;
generating an alert if the medication was administered at a wrong time or by an unqualified person.

28. A method according to claim 24, further comprising:
receiving inventory information describing inventory of medication at the pharmacy or care facility;
comparing the inventory information with prescription information or a threshold percent of medication stock at the pharmacy or care facility; and
selectively submitting an order for medication based on the comparison.

29. A computer readable medium having computer executable instructions for performing the method of claim 24.
